(19) United States
(12) Patent Application Publication
(10) Pub. No.: US 2021/0199640 A1
(43) Pub. Date: Jul. 1, 2021

(54) Methods of Normalizing Aberrant Glycolytic Metabolism in Cancer Cells

(71) Applicant: The Board of Trustees of the Leland Stanford Junior Unviersity, Stanford, CA (US)

(72) Inventors: Chirag Bihesh PATEL, Palo Alto, CA (US); Corinne G. Beinat, Redwood City, CA (US); Edwin Chang, Menlo Park, CA (US); Sanjiv Sam Gambhir, Portola Valley, CA (US); Arutselvan Natarajan, Sunnyvale, CA (US)

(21) Appl. No.: 17/133,853

(22) Filed: Dec. 24, 2020

Related U.S. Application Data
(60) Provisional application No. 62/953,704, filed on Dec. 26, 2019.

(51) Int. Cl. G01N 33/487

(52) U.S. Cl. CPC G01N 33/48707; A61K 33/243

(57) ABSTRACT

Viability of cancer cells (e.g., glioblastoma cells) can be reduced by administering mannose to the cancer cells; and applying an alternating electric field with a frequency between 100 and 500 kHz to the cancer cells. Susceptibility to treatment with an alternating electric field can be determined by measuring uptake of a PKM2 probe (e.g., [18F]DASA) before and after treatment with an alternating electric field. Notably, experiments show that the combination of mannose and the alternating electric field produces a synergistic anti-glioblastoma result.

### CROSS REFERENCE TO RELATED APPLICATIONS

This application claims the benefit of U.S. Provisional Application 62/953,704, filed Dec. 26, 2019, which is incorporated herein by reference in its entirety.

### BACKGROUND

Tumor Treating Fields (TTFields) are an effective anti-neoplastic treatment modality delivered via non-invasive application of low intensity, intermediate frequency (e.g., 100-500 kHz), alternating electric fields. TTFields exert directional forces on polar microtubules and interfere with the normal assembly of the mitotic spindle. Such interference with microtubule dynamics results in abnormal spindle formation and subsequent mitotic arrest or delay. Cells can die while in mitotic arrest or progress to cell division leading to the formation of either normal or abnormal aneuploid progeny. The formation of tetraploid cells can occur either due to mitotic exit through slippage or can occur during improper cell division. Abnormal daughter cells can die in the subsequent interphase, can undergo a permanent arrest, or can proliferate through additional mitosis where they will be subjected to further TTFields assault. Giladi M et al. Sci Rep. 2015; 5:18046.

In the in vivo context, TTFields therapy can be delivered using a wearable and portable device)(Optune®). The delivery system includes an electric field generator, 4 adhesive patches (non-invasive, insulated transducer arrays), rechargeable batteries and a carrying case. The transducer arrays are applied to the skin and are connected to the device and battery. The therapy is designed to be worn for as many hours as possible throughout the day and night.

In the preclinical setting, TTFields can be applied in vitro using, for example, the Inovitro™ TTFields lab bench system. Inovitro™ includes a TTFields generator and base plate containing 8 ceramic dishes per plate. Cells are plated on a 22 mm round cover slip placed inside each dish. TTFields are applied using two perpendicular pairs of transducer arrays insulated by a high dielectric constant ceramic in each dish. The orientation of the TTFields in each dish is switched 90° every 1 second, thus covering different orientation axes of cell divisions.

Pyruvate kinase M2 (PKM2) is a key marker of cancer metabolic reprogramming since it catalyzes the final step in glycolysis. 1-((2-fluoro-6-[18F]fluorophenyl)sulfonyl)-4-((4-methoxyphenyl)sulfonyl)piperazine (hereinafter [18F]DASA-23) is a radiotracer that measures aberrantly-expressed PKM2 in glioblastoma (GBM). Approved therapeutic modalities for treating tumors such as GBM include surgery, temozolomide (TMZ) chemotherapy, radiotherapy, and TTFields. And there is an important need to assess early on whether a patient's GBM is responding to a given therapy (e.g., TTFields therapy).

Mannose is a monosaccharide that has been shown to inhibit tumor growth in vitro and in vivo. Gonzalez et al., Mannose impairs tumour growth and enhances chemotherapy, Nature, volume 563, pp 719-723 (2018). Mannose and glucose share transporters responsible for uptake into cells. Id.

### SUMMARY

The inventors have determined that (1) a PKM2 probe (e.g., [18F]DASA-23, etc.) can be used to determine susceptibility of patients to treatment of glioblastoma with TTFields, and (2) treating tumors (e.g., glioblastoma) with the combination of mannose and TTFields provides a synergistic result. The inventors have also determined that labelled mannose can be used as a probe for detecting changes in glioblastoma metabolism and determining susceptibility of patients to treatment of glioblastoma with TTFields and mannose.

Aspects described herein provide methods of determining susceptibility of patient to treatment of cancer (e.g., glioblastoma) with alternating electric fields comprising: administering a PKM2 probe to a patient having cancer; measuring a first level of PKM2 uptake in cancer cells of the patient; exposing the cancer cells to treatment using alternating electric fields at a frequency between 100 and 500 kHz after measuring the first level; measuring a second level of PKM2 uptake in the cancer cells; and determining if the patient is susceptible to treatment using alternating electric fields based on whether the first level is higher than the second level by at least 5%.

Further aspects described herein provide methods of reducing a viability of cancer cells (e.g. glioblastoma cells), by administering a PKM2 probe to a patient having cancer; measuring a first level of PKM2 expression or uptake of the PKM2 probe in cancer cells from the patient; exposing the cancer cells to alternating electric fields at a frequency between 100 and 500 kHz for a first interval of time after measuring the first level; measuring a second level of PKM2 expression or uptake of the PKM2 probe in the cancer cells after the first interval of time; and continuing exposing the cancer cells to alternating electric fields if the first level is higher than the second level by at least 5%.

Further aspects described herein provide methods of determining susceptibility of patient to treatment of cancer (e.g., glioblastoma) using alternating electric fields by administering mannose labelled with an imaging probe to cells of patient having cancer; measuring a first level of uptake of the mannose labelled with an imaging probe in cancer cells from the patient; treating the cancer cells with alternating electric fields at a frequency between 100 and 500 kHz for a first interval of time after measuring the first level; measuring a second level of uptake of the mannose labelled with an imaging probe in the cancer cells after the first interval of time; and continuing treatment of the cancer cells using alternating electric fields if the first level is lower than the second level by at least 10%.

Yet further aspects described herein provide methods of reducing a viability of cancer cells (e.g., glioblastoma cells), comprising administering mannose to cancer cells of a patient having cancer and then exposing the cancer cells to alternating electric fields at a frequency between 100 and 500 kHz.

Aspects described herein provide methods of reducing a viability of cancer cells (e.g., glioblastoma cells), by administering a PKM2 probe to a patient having cancer; measuring a first level of PKM2 expression or uptake of the PKM2 probe in cancer cells from the patient; exposing the cancer cells to alternating electric fields at a frequency between 100 and 500 kHz for a first interval of time after measuring the first level; measuring a second level of PKM2 expression or uptake of the PKM2 probe in the cancer cells after the first interval of time; and administering a chemotherapeutic agent to the cancer cells if the first level is higher than the second level by at least 5%.

### BRIEF DESCRIPTION OF THE DRAWINGS

FIG. 1 illustrates an application of Tumor Treating Fields (“TTFields”) to treatment of glioblastoma (GBM);

FIG. 2 illustrates the use of TTFields to prolong survival in GBM patients alone and in combination with chemotherapy;

FIG. 3 illustrates the differences in glycolysis between normal tissue (oxidative phosphorylation) and tumor tissue (Warburg effect);

FIG. 4 illustrates how TTFields causes a shift in GBM glycolysis as measured by regulation of PKM2 using [18F]DASA-23 as a measurement tracer;

FIG. 5 illustrates an exemplary method to measure the effects of TTFields on uptake of [18F]DASA-23 in GBM cells (e.g., U87, GBM39);

FIG. 6 shows that PKM2 expression is reduced in GBM after chemotherapy standard of care (TMZ) or TTFields in U87 cells;

FIG. 7 shows that PKM2 expression is reduced in GBM after exposure to TTFields in U87 cells as shown by reduced uptake of [18F]DASA-23;

FIG. 8 shows that PKM2 expression is reduced in GBM after exposure to TTFields in U87 cells as shown by Western blot for the PKM2 protein;

FIG. 9 shows that TTFields exposure reduced PKM2 expression in U87 cells as shown by immunofluorescence;

FIG. 10 shows the effect on total cell number by treatment with mannose on U87 cells with and without TTFields treatment based on the counting of viable cells via a hemocytometric technique;

FIG. 11 shows the effect on percent cell count with respect to no mannose on U87 cells with and without TTFields treatment based on the counting of viable cells via a hemocytometric technique;

FIG. 12A shows the results of an exemplary experiment demonstrating that TTFields application reduces the levels of the PKM2 protein as shown in a western blot of cell lysates from control OVCAR3 human ovarian adenocarcinoma cells compared to cells treated with TTFields for 72 hours, cells treated with cisplatin, and cells treated with cisplatin and TTFields; and

FIG. 12B quantifies and presents the data of FIG. 12A in a bar graph format.

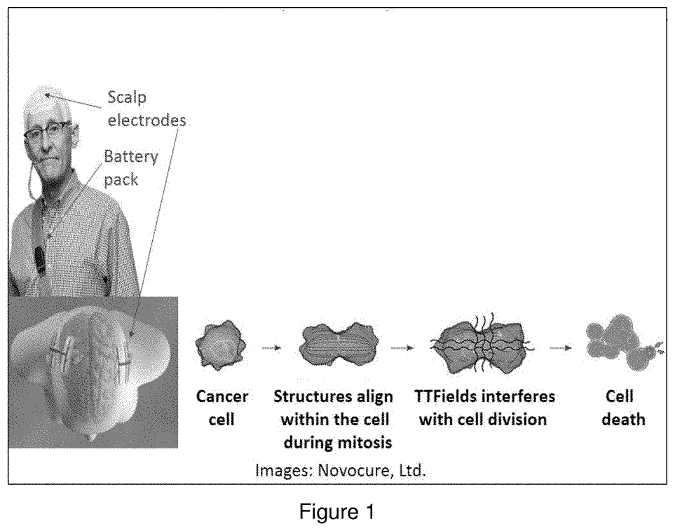

### DETAILED DESCRIPTION OF THE PREFERRED EMBODIMENTS

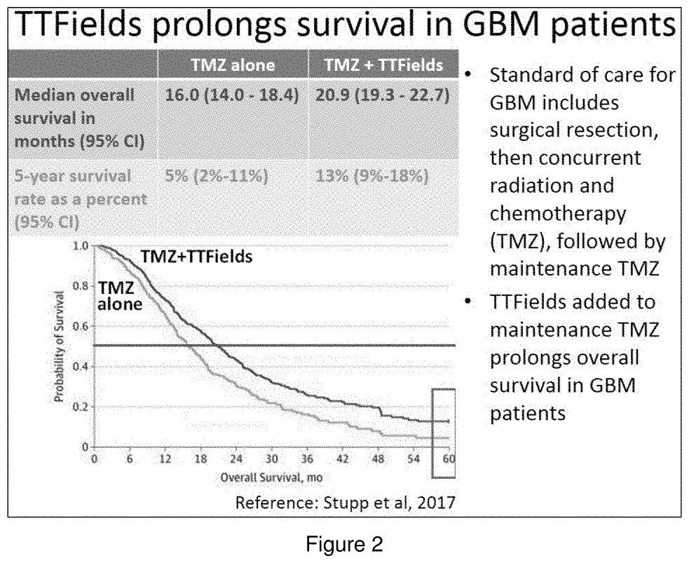

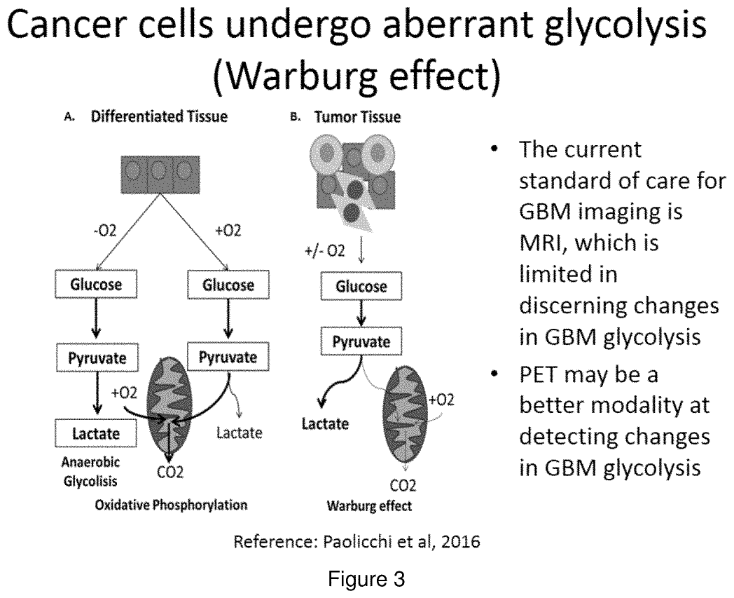

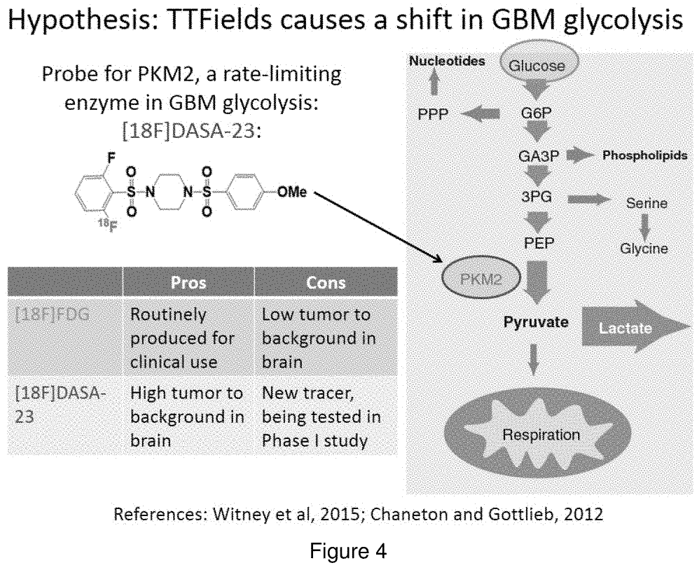

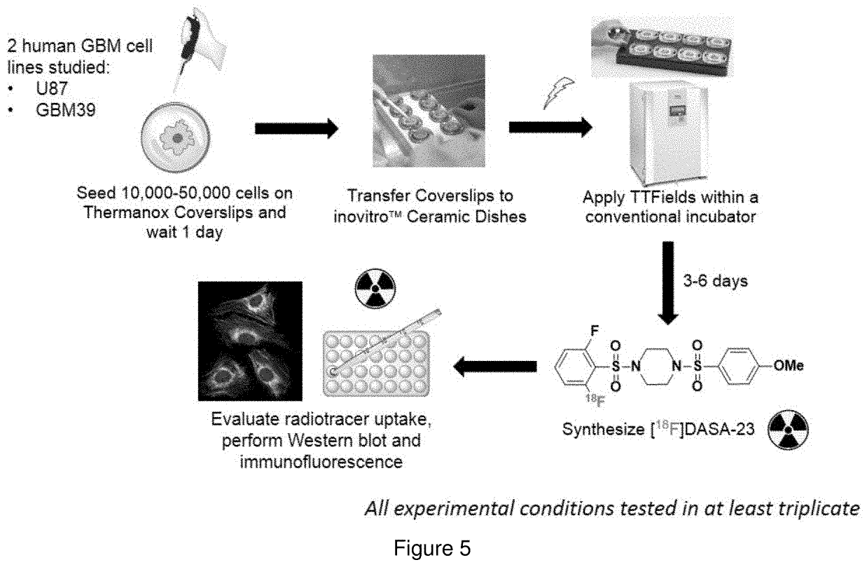

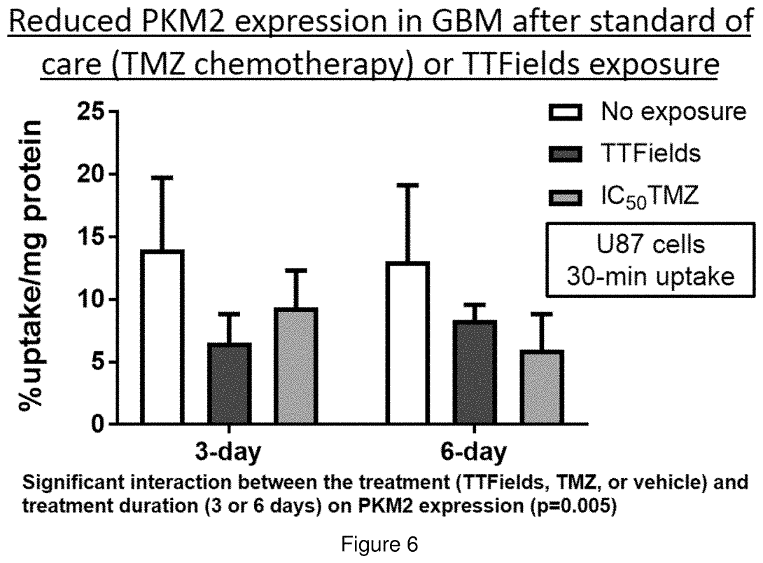

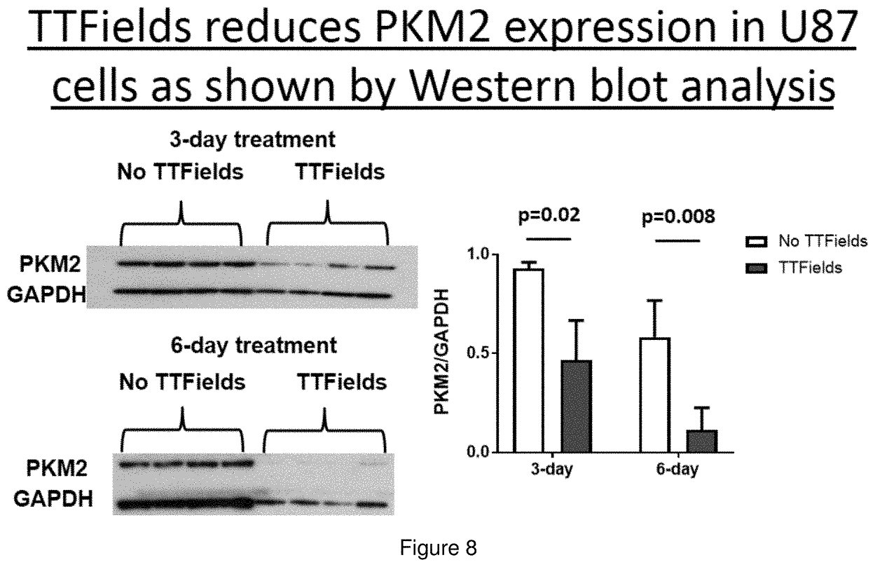

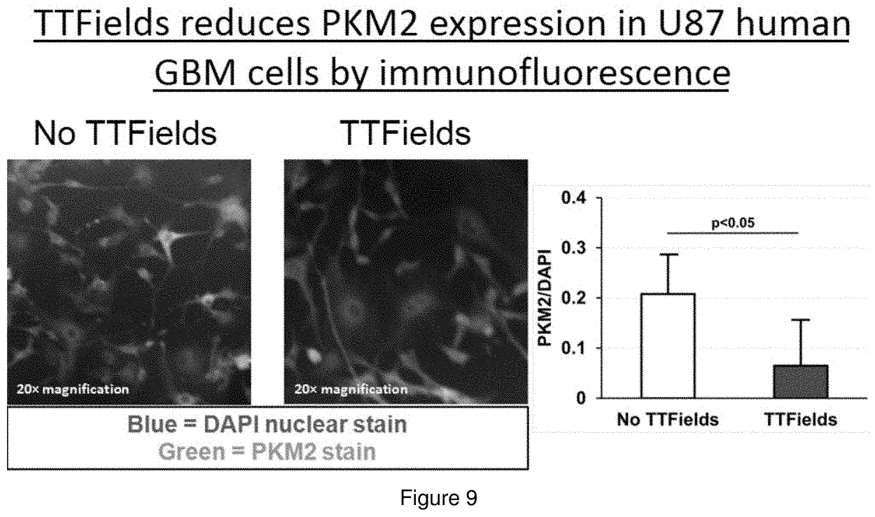

All references cited herein, including but not limited to patents and patent applications, are incorporated herein by reference in their entirety.

[18F]DASA-23 can be used to detect changes in GBM metabolism in response to TMZ and TTFields therapies. In one experiment, Human U87 GBM cells were subjected to 200 kHz TTFields, the IC₅₀ of TMZ, or vehicle for three or six days (n≥3/condition), followed by evaluation of [18F]DASA-23 uptake (e.g., FIGS. 2 and 6). Immunofluorescence for PKM2 was performed to confirm the [18F]DASA-23 uptake results. (e.g., FIG. 9). Western blot analysis was performed to determine the effect of TMZ and TTFields exposure on the expression of PKM2. 2-way ANOVA with multiple comparisons was performed. (e.g., FIG. 8). Data are reported as mean±SD.

TTFields reduces PKM2 expression in GBM, indicating a shift from aberrant glycolysis (i.e., Warburg effect) towards oxidative phosphorylation. PKM2 expression is a biomarker of this shift, as validated by radiotracer uptake, Western blot, and immunofluorescence assays. (e.g., FIGS. 2, 6-9). Aspects described herein provide for non-invasive assessment of GBM's glycolytic response to various therapies using [18F]DASA-23.

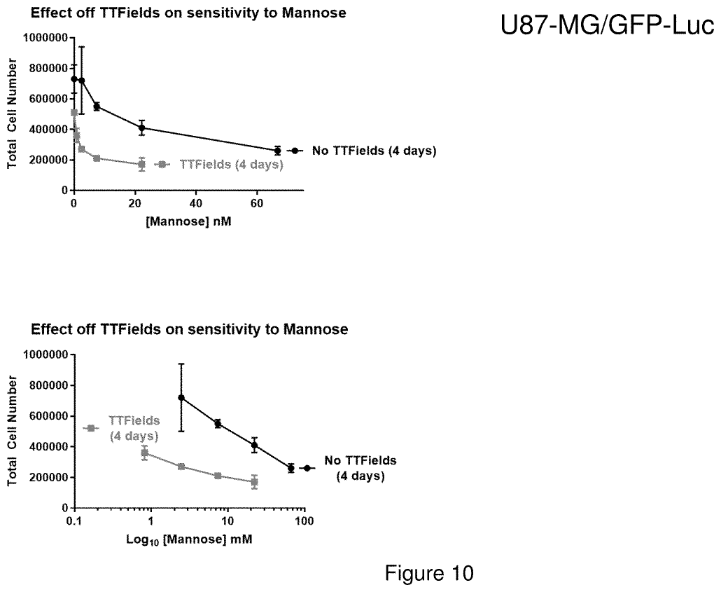

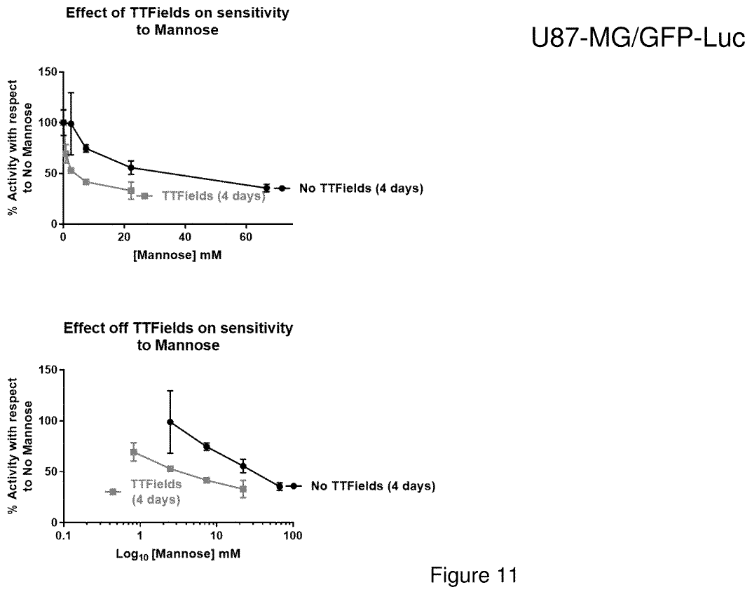

Further aspects provide methods of inhibiting the growth of GBM cells by administering mannose and TTFields to glioblastoma cells of a patient in need of treatment resulting in synergistic inhibition of GBM cells. (e.g., FIGS. 10 and 11).

An important feature of cancer cells is the metabolic curiosity known as the Warburg effect whereby tumor cells prefer fermentation as a source of energy rather than the more efficient mitochondrial pathway of oxidative phosphorylation (OxPhos), even in the presence of oxygen. Normal tissues only use this less efficient pathway in the absence of oxygen. From a biochemical standpoint, this reprogramming of metabolism is manifested at several stages in the glycolytic pathway. Prominent amongst them are changes in expression and distribution of membrane-associated glucose transporters as well as alterations in the activity and expression of the principal enzyme involved with the final step of glycolysis, namely, pyruvate kinase.

The [18F]DASA-23 radiotracer has been developed to measure the expression of PKM2 while [18F] Deoxyglucose ([18F]-FDG) is used to monitor enhanced glucose uptake into cancer cells. Glioblastoma (GBM) is traditionally treated with surgical resection, temozolomide (TMZ) chemotherapy, and/or radiation therapy. Tumor treating fields (TTFields), i.e., the application of alternating electric fields (e.g., 100-500 kHz, 1-4 V/cm) to tumors, is a fourth approved therapeutic modality in GBM. There is an important need to assess early on whether a patient's GBM is responding to a given therapy, including but not limited to TTFields therapy.

The ability of [18F]DASA-23 to detect changes in metabolism in response to TMZ and TTFields therapies, in cell culture and an orthotopic murine model of human GBM (FIG. 6) was evaluated. There was a significant interaction between the treatment (vehicle, TMZ, or TTFields) and treatment duration (3 or 6 days) on PKM2 expression as measured by cellular uptake of [18F]DASA-23 (p=0.005, 2-way ANOVA) (FIG. 6).

Immunofluorescence for PKM2 in TTFields-exposed and unexposed U87-MG cells revealed reduced cell count and less intense PKM2 staining due to TTFields.

Mannose and TTFields Synergistically Interact to Reduce Viability of GBM Cells

Mannose is known to occupy the same transporter system as that of glucose. As such, it acts as a competitive inhibitor to glucose for the glucose transporter. It has been shown to inhibit the glycolytic metabolism of glucose, and thus directly affect cellular growth by altering cancer metabolism. The inventors performed combination interventions with mannose and TTFields and have shown a significant, synergistic interaction between the two interventions in terms of reduction in glioblastoma cell count.

The mannose+TTFields data (FIGS. 10 and 11) suggest that TTFields affects metabolic pathways that involve glucose and mannose uptake. Without being bound by this theory, it is believed that TTFields induces a shift from aberrant glycolysis (the so-called “Warburg effect”) to normal oxidative phosphorylation. In one aspect, mannose labelled with an imaging probe could have both diagnostic and therapeutic effects (i.e., a theranostic). Mannose+TTFields data was generated as follows:

Growth Conditions for Human Glioblastoma Cells

U87-MG and MDA-MB-231 were grown in DMEM (Invitrogen/Life Technologies, Carlsbad, Calif., USA/10% FBS/ and 1× Antibiotic-Antimycotic) and 1×antibiotic/anti-mycocytic agents. GBM2 and GBM39 were grown in a defined, serum-free media of a 1:1 mixture of Neurobasal-A Medium (1×)/DMEM/F12(1×) that also contained HEPES Buffer Solution (10 mM), MEM Sodium Pyruvate Solution 1 mM, MEM Non-Essential Amino Acids Solution 10 mM (1×), GlutaMAX-I Supplement (1×) and Antibiotic-Antimycotic (1×). These solutions were obtained from Invitrogen/Life Technologies Inc. (Carlsbad, Calif., USA). The full working media also contained H-EGF (20 ng/mL), H-FGF-basic-154 (20 ng/mL), H-PDGF-AA (10 ng/mL), H-PDGF-BB (10 ng/mL) and Heparin Solution, 0.2% (2 μg/mL) as growth factors (all from Shenandoah Inc., Warwick, Pa., USA) and B-27 (Invitrogen/Life Technologies, Carlsbad, Calif., USA) as supplements.

Growth Experiments with Inovitro™ System

In this aspect, 50,000 single cells were suspended in 200 μL of media and seeded in the middle of a 22 mm diameter cover slip. The cover slips were placed in a 6-well plate and allowed to incubate in a conventional tissue culture incubator (37° C., 95% air, 5% CO₂) overnight. Once cells adhered to the cover slip, an additional 2 mL of media was added to each well. The cells remained on the cover slips for 2-3 days in order to achieve the growth phase, before they were transferred to a ceramic dish of the Inovitro™ system, which in turn was mounted onto Inovitro™ base plates (Novocure Inc., Haifa, Israel). TTFields set anywhere from 1-4 V/cm were applied through an Inovitro™ power generator while frequencies ranged from 50-500 kHz. Incubation temperatures spanned 20-27° C. with a target temperature of 37° C. for the ceramic dishes upon application of the TTFields. The culture was incubated for a control period of 24 h before treatment. Treatment duration lasted anywhere from 1-6 days, after which cover slips were removed and cell counts per cover slip were determined. Culture medium was exchanged manually every 24 h throughout the experiments. Corresponding control experiments were done by placing equivalent cover slips within ceramic dishes into a conventional tissue culture incubator (37° C., 5% CO₂) and cells grown in parallel with the TTField-exposed coverslips. Unless otherwise mentioned, all experiments per condition were done in triplicate samples with four measurements (cell counts) per sample.

The term “[18F]DASA-23” refers to 1-((2-fluoro-6-[18F]fluorophenyl)sulfonyl)-4-((4-methoxyphenyl)sulfonyl)piperazine having the following chemical structure:

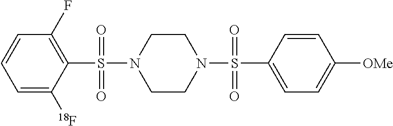 [SMILES: COc1ccc(S(=O)(=O)N2CCN(S(=O)(=O)c3c(F)cccc3[18F])CC2)cc1]

and pharmaceutically acceptable salts thereof. [18F]DASA-23 can be used in combination with a pharmaceutically acceptable carrier for administration to a patient.

The term “reducing viability of cancer cells” or “reducing viability of glioblastoma cells” as used herein, refers to reducing the growth, proliferation, or survival of the cancer cell (e.g., GBM cell). In some aspects, the reduction in viability of the cancer cells comprises reducing clonogenic survival of the cancer cells, increasing cytotoxicity of the cancer cells, inducing apoptosis in the cancer cells, and decreasing tumor volume in a tumor formed from at least a portion of the cancer cells.

Aspects described herein provide methods of determining susceptibility of a patient to treatment of cancer (e.g., glioblastoma) with TTFields (e.g., alternating electric fields) comprising administering a PKM2 probe to a patient having cancer, measuring a first level of PKM2 uptake in cancer cells from the patient, exposing the cancer cells to TTField treatment (e.g., alternating electric fields), measuring a second level of PKM2 uptake in the cancer cells, and determining if a patient is susceptible to treatment with TTFields based on whether the first level is higher than the second level by at least 5% (e.g., at least 5, 10, 15, 20, 30, 40, 50, 60, 70, 80, or 90%). In this aspect, a decrease in the level of PKM2 uptake indicate a change in metabolism which makes cancer cells (e.g., glioblastoma) susceptible to treatment with TTFields.

A PKM2 probe can be administered to a patient by any suitable method known in the art (e.g., injection, oral administration, and ex vivo). In some instances, glioblastoma or tumor cells can be removed from a patient (e.g., by a biopsy) and the measurement of PKM2 expression or uptake can be made in cell culture, for example, before and after exposure to TTFields.

In some instances, the PKM2 probe comprises [18F]DASA-23 having the following structure:

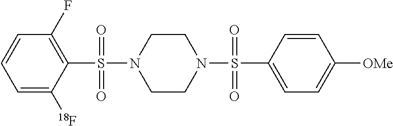 [SMILES: COc1ccc(S(=O)(=O)N2CCN(S(=O)(=O)c3c(F)cccc3[18F])CC2)cc1]

In some instances, the alternating electric fields have a frequency between 180 and 220 kHz. In some instances, the cancer is a brain cancer such as glioblastoma.

Aspects described herein provide methods of reducing a viability of cancer cells, by administering a PKM2 probe to a patient having cancer; measuring a first level of PKM2 expression or uptake of the PKM2 probe in cancer cells from the patient; exposing the cancer cells to alternating electric fields at a frequency between 100 and 500 kHz for a first interval of time after measuring the first level; measuring a second level of PKM2 expression or uptake of the PKM2 probe in the cancer cells after the first interval of time; and continuing exposing the cancer cells to alternating electric fields if the first level is higher than the second level by at least 5%.

In some instances, PKM2 probe comprises [18F]DASA-23 having the structure provided herein. In some instances, the alternating electric fields have a frequency between 180 and 220 kHz. In some instances, the cancer cells are glioblastoma cells.

Aspects described herein provide methods of determining susceptibility of patient to treatment of cancer using alternating electric fields by administering mannose labelled with an imaging probe to cells of patient having cancer; measuring a first level of uptake of the mannose labelled with an imaging probe in cancer cells from the patient; treating the cancer cells with alternating electric fields at a frequency between 100 and 500 kHz for a first interval of time after measuring the first level; measuring a second level of uptake of the mannose labelled with an imaging probe in the cancer cells after the first interval of time; and continuing treatment of the cancer cells using alternating electric fields if the first level is lower than the second level by at least 10%.

In some instances, the alternating electric fields have a frequency between 180 and 220 kHz. In some instances, the cancer is glioblastoma.

Aspects described herein provide methods of reducing a viability of cancer cells, comprising administering mannose to cancer cells of a patient having cancer and then exposing the cancer cells to alternating electric fields at a frequency between 100 and 500 kHz. In some instances, the alternating electric fields have a frequency between 180 and 220 kHz. In some instances, the cancer cells are brain cancer cells such as glioblastoma cells.

Aspects described herein provide methods of reducing a viability of cancer cells, by administering a PKM2 probe to a patient having cancer; measuring a first level of PKM2 expression or uptake of the PKM2 probe in cancer cells from the patient; exposing the cancer cells to alternating electric fields at a frequency between 100 and 500 kHz for a first interval of time after measuring the first level; measuring a second level of PKM2 expression or uptake of the PKM2 probe in the cancer cells after the first interval of time; and administering a chemotherapeutic agent to the cancer cells if the first level is higher than the second level by at least 5%.

In some instances, the chemotherapeutic agent is selected from the group consisting of tamoxifen, cisplatin, 5-fluorouracil (5-FU), and docetaxel. In some instances, the chemotherapeutic agent is cisplatin.

Further aspects comprise continuing exposing the cancer cells to alternating electric fields. In some instances, the cancer cells are glioblastoma cells

As shown in FIG. 6, PKM2 expression in U87 glioblastoma cells over a 30 minute period is reduced by at least 10% after treatment with chemotherapy (TMZ—Temozolomide) and by at least 10% after exposure to TTFields.

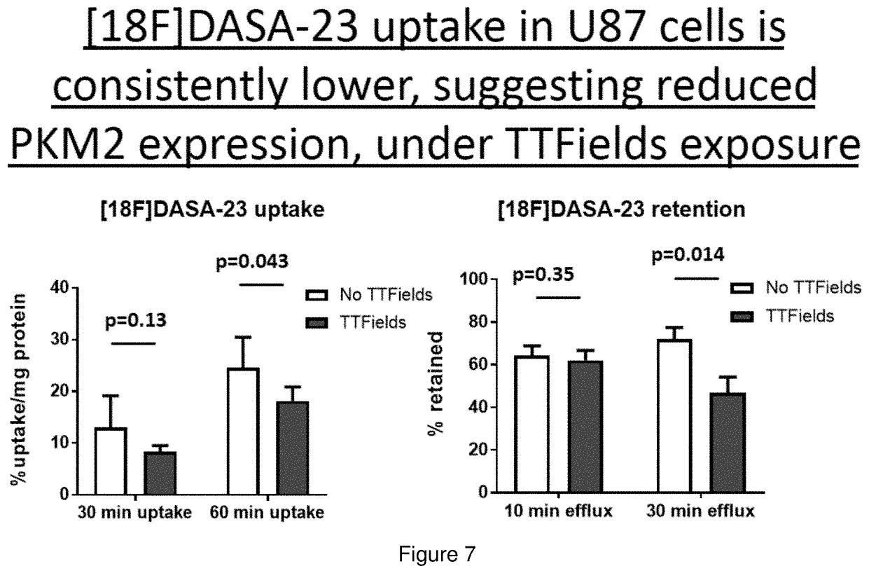

FIG. 7 shows that after exposure to TTFields (1) [18F]DASA-23 uptake is reduced by at least 5% over 30 minutes and at least 10% over 60 minutes in U87 cells and (2) [18F]DASA-23 retention is reduced by at least 20% over 60 minutes.

FIG. 8 shows that exposure to TTFields reduces PKM2 expression in U87 cells by Western blot by about 50% after 3 days and about 80% after 6 days.

FIG. 9 shows that exposure to TTFields reduces PKM2 expression in U87 cells by immunofluorescence with blue (dark) showing DAPI nuclear stain and green (light) showing PKM2. PKM2 expression is reduced by more than 50%.

As shown in FIGS. 10 and 11, there is an IC50 difference of about 10-fold between treatment with mannose alone compared to mannose with TTFields. Without being bound by theory, it is believed that there would be about a 5-fold reduction of this effect in vivo and through use of, for example, a mannose PET (positron emission tomography) probe. Therefore, it is believed that about a 20% increase in mannose uptake would indicate a metabolic treatment response to mannose plus TTFields.

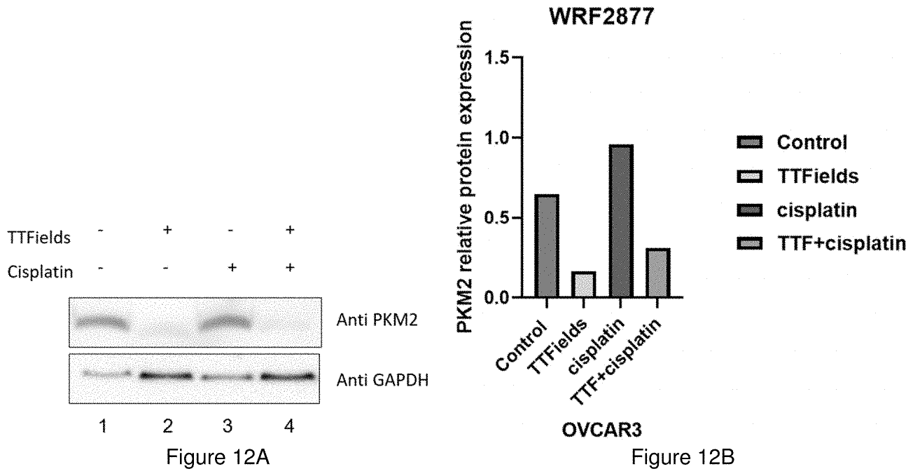

As shown in FIGS. 12A-12B, application of TTFields decreases PKM2 protein levels. FIGS. 12A and 12B show the results of an exemplary experiment comparing levels of PKM2 protein in a Western blot of lysates produced from control OVCAR3 human ovarian adenocarcinoma cells as between (1) cells that have undergone TTFields application for 72 hours (2), Cisplatin 300 nM (3) a combination of Cisplatin and TTFields (4) and a control. As seen in FIG. 12A, Western blot results were quantified relative to housekeeping gene expression (GAPDH).

The OVCAR-3 cell line was obtained from ATCC. Cells were cultured in ATCC-formulated RPMI-1640 Medium, Catalog No. 30-2001 supplemented with 0.01 mg/ml bovine insulin; 20% fetal bovine and antibiotics. 30,000 single cells were suspended in 500 μL of media and seeded in the middle of a 22 mm diameter cover slip.

For induction of 72 hours TTFields application, the cover slips were placed in a ceramic dish of Inovitro™ system and allowed to incubate in a conventional tissue culture incubator (37° C., 95% air, 5% CO₂) overnight. Once cells adhered to the cover slip, an additional 1.5 mL of media were added to each well and covered in Parafilm (P7793, Sigma Aldrich) to avoid evaporation of media. Cisplatin was added to a final concentration of 300 nM. After an overnight incubation, dishes were mounted onto Inovitro™ base plates (Novocure Inc., Haifa, Israel). TTFields set anywhere from 1-6 V/cm were applied through an Inovitro™ power generator while frequencies were 200 kHz. Incubation temperature was 18° C. with a target temperature of 37° C. for the ceramic dishes upon application of the TTFields. Corresponding control experiments were done by placing equivalent cover slips within ceramic dishes into a conventional tissue culture incubator (37° C., 5% CO₂) and cells grown in parallel with the TTFields-exposed coverslips.

Cell Lysates and Immunoblotting

Following TTFields application, cells were transferred to cold PBS plates for wash.

RIPA lysis buffer (R0278, Sigma-Aldrich), supplemented with a cocktail of protease (Complete Mini, Roche), and phosphatase inhibitors (Halt #78420, Thermo Scientific) was added to plates and cells were scraped with approximately 100 μl supplemented RIPA buffer for 8 Inovitro™ dishes.

Extracts were shaken at 4° C. for a duration of 30 minutes. Samples were centrifuged (20 min, 14,000 rpm, 4° C.). Supernatant was transferred and protein concentration was determined by BCA protein assay kit (BCA protein assay kit, ab102536 Abcam).

After determining protein concentration, 30 μg protein were resolved under reducing conditions (Bolt Sample reducing agent, #2060435 and Sample buffer #2045289, Novex) and samples were boiled at 100° C. for 5 minutes. Samples were run on SDS-polyacrylamide gel electrophoresis (Bolt 8% Bis-Tris base gel NW00080BOX, Thermo-Fischer).

After electrophoresis, proteins were transferred to 0.2 μm polyvinylidene difluoride membrane (Immuno-Blot PVDF #162-0177, Bio-Rad) and probed with the appropriate primary antibody: GAPDH (SC-32233, Santa Cruz), and PKM2 (ab137852, abcam), followed by horseradish peroxidase-conjugated secondary antibody (goat anti rabbit 7074, Cell Signaling and goat anti mouse 7076, Cell Signaling) and a chemiluminescent substrate (WBLUF0100, Signa-Aldrich). Quantification of bands was done by Image J software.

In another aspect, methods of reducing the viability of glioblastoma cells, comprising administering mannose to cells of patient having glioblastoma and exposing the glioblastoma cells to TTFields are provided.

In any of the aspects described above, the PKM2 probe may optionally comprise [18F]DASA-23 having the following structure:

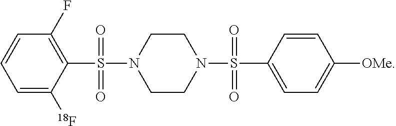 [SMILES: COc1ccc(S(=O)(=O)N2CCN(S(=O)(=O)c3c(F)cccc3[18F])CC2)cc1]

Aspects described herein provide methods of delivering mannose to cancer cells (e.g., GBM cells) at a therapeutically effective concentration, wherein the alternating electric field has a field strength of at least 1 V/cm in at least some of the cancer cells.

The term “therapeutically effective concentration,” as used herein, refers to the concentration of mannose sufficient to achieve its intended purpose (e.g., treatment of cancer, treatment of GBM). In one aspect, a therapeutically effective concentration of mannose is between 1 to 10 mM.

In another aspect, the step of applying an electrical field has a duration of at least 72 hours. The application of the electrical field for 72 hours may be accomplished in a single 72 hour interval. Alternatively, the application of the electrical field could be interrupted by breaks. For example, 6 sessions with a duration of 12 hours each, with a 2 hour break between sessions. In another aspect, the step of applying an electrical field has a duration of at least 4 hours.

In yet another aspect, the frequency of the alternating electric field is between 180 and 220 kHz. In another aspect, the mannose is delivered to the cancer cells at a therapeutically effective concentration, and the alternating electric field has a field strength of at least 1 V/cm in at least some of the cancer cells.

In yet another aspect, at least a portion of the applying step is performed simultaneously with at least a portion of the administering step.

In a further aspect, the mannose is delivered to the cancer cells at a therapeutically effective concentration, and the alternating electric field has a field strength of at least 1 V/cm in at least some of the cancer cells. Optionally, the applying step has a duration of at least 72 hours and the frequency of the alternating electric field is between 180 and 220 kHz. Optionally, at least a portion of the applying step can be performed simultaneously with at least a portion of the administering step.

Without being bound by theory, it is believed that lower uptake of [18F]DASA-23 correlates with reduced activity of PKM2, and indeed, the western blot analysis of protein samples from treated cells has revealed lower expression of PKM2 post treatment (FIGS. 8, 12A-12B). It has been shown that following TTFields application in glioma cells, there is a significantly lower uptake of [18F]DASA-23. It is believed that reducing PKM2 expression sensitizes cancer cells to different cytotoxic agents where resistance to treatment was shown to be associated with increased PKM2 activity. Ji et al., Tumor Biology, June 2017: 1-11; Gao et al., J Cancer Res Clin Oncol. 2011 January; 137(1):65-72; Shin et al., Electrophoresis, Volume 30, Issue 12, June 2009, Pages 2182-2192; Shi et al., Cancer Science, Volume 101, Issue 6, June 2010, Pages 1447-1453. Combining TTFields treatment with chemotherapeutic agents after, for example, reducing PKM2 expression, could increase treatment efficacy and decrease the therapeutically effective dose of the chemotherapeutic agent.

The in vitro experiments described herein were carried out using the Novocure Inovitro™ system. In these experiments, the direction of the alternating electric fields was switched at one second intervals between two perpendicular directions. But in alternative embodiments, the direction of the alternating electric fields can be switched at a faster rate (e.g., at intervals between 1 and 1000 ms) or at a slower rate (e.g., at intervals between 1 and 100 seconds).

In the in vitro experiments described herein, the direction of the alternating electric fields was switched between two perpendicular directions by applying an AC voltage to two pairs of electrodes that are disposed 90° apart from each other in 2D space in an alternating sequence. But in alternative embodiments the direction of the alternating electric fields may be switched between two directions that are not perpendicular by repositioning the pairs of electrodes, or between three or more directions (assuming that additional pairs of electrodes are provided). For example, the direction of the alternating electric fields may be switched between three directions, each of which is determined by the placement of its own pair of electrodes. Optionally, these three pairs of electrodes may be positioned so that the resulting fields are disposed 90° apart from each other in 3D space. In other alternative embodiments, the electrodes need not be arranged in pairs. See, for example, the electrode positioning described in U.S. Pat. No. 7,565,205, which is incorporated herein by reference. In other alternative embodiments, the direction of the field remains constant.

In the in vitro experiments using the Inovitro™ system described herein, the electrical field was capacitively coupled into the culture because the Inovitro™ system uses conductive electrodes disposed on the outer surface of the dish sidewalls, and the ceramic material of the sidewalls acts as a dielectric. But in alternative embodiments, the electric field could be applied directly to the cells without capacitive coupling (e.g., by modifying the Inovitro™ system configuration so that the conductive electrodes are disposed on the sidewall's inner surface instead of on the sidewall's outer surface).

The methods described herein can also be applied in the in vivo context by applying the alternating electric fields to a target region of a live subject's body (e.g., using the Novocure Optune® system). This may be accomplished, for example, by positioning electrodes on or below the subject's skin so that application of an AC voltage between selected subsets of those electrodes will impose the alternating electric fields in the target region of the subject's body.

For example, in situations where the relevant cells are located in the subject's brain, one pair of electrodes could be positioned on the front and back of the subject's head, and a second pair of electrodes could be positioned on the right and left sides of the subject's head. In some embodiments, the electrodes are capacitively coupled to the subject's body (e.g., by using electrodes that include a conductive plate and also have a dielectric layer disposed between the conductive plate and the subject's body). But in alternative embodiments, the dielectric layer may be omitted, in which case the conductive plates would make direct contact with the subject's body. In another embodiment, electrodes could be inserted subcutaneously below a patient's skin. An AC voltage generator applies an AC voltage at a selected frequency (e.g., 200 kHz) between the right and left electrodes for a first period of time (e.g., 1 second), which induces alternating electric fields where the most significant components of the field lines are parallel to the transverse axis of the subject's body.

Then, the AC voltage generator applies an AC voltage at the same frequency (or a different frequency) between the front and back electrodes for a second period of time (e.g., 1 second), which induces alternating electric fields where the most significant components of the field lines are parallel to the sagittal axis of the subject's body. This two step sequence is then repeated for the duration of the treatment. Optionally, thermal sensors may be included at the electrodes, and the AC voltage generator can be configured to decrease the amplitude of the AC voltages that are applied to the electrodes if the sensed temperature at the electrodes gets too high. In some embodiments, one or more additional pairs of electrodes may be added and included in the sequence. In alternative embodiments, only a single pair of electrodes is used, in which case the direction of the field lines is not switched. Note that any of the parameters for this in vivo embodiment (e.g., frequency, field strength, duration, direction-switching rate, and the placement of the electrodes) may be varied as described above in connection with the in vitro embodiments. But care must be taken in the in vivo context to ensure that the electric field remains safe for the subject at all times.

Note that in the experiments described herein, the TTFields were applied for an uninterrupted interval of time (e.g., 72 hours or 14 days). But in alternative embodiments, the application of TTFields may be interrupted by breaks that are preferably short. For example, a 72 hour interval of time could be satisfied by applying the alternating electric fields for six 12 hour blocks, with 2 hour breaks between each of those blocks.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the claims listed below, and equivalents thereof.

### REFERENCES

1. Giladi M et al. Sci Rep. 2015; 5:18046
2. Gonzalez et. al., Nature volume 563, pages 719-723 (2018).
3. Ji et al., Tumor Biology, June 2017: 1-11.
4. Guo et al., J Cancer Res Clin Oncol. 2011 January; 137(1):65-72.
5. Shin et. al., Electrophoresis, June 2009, Pages 2182-2192.
6. Shi et al., Cancer Science, Volume 101, Issue 6, June 2010, Pages 1447-1453.

What is claimed is:

1. A method of determining susceptibility of patient to treatment of cancer with alternating electric fields comprising:
administering a PKM2 probe to a patient having cancer;
measuring a first level of PKM2 uptake in cancer cells of the patient;
exposing the cancer cells to treatment using alternating electric fields at a frequency between 100 and 500 kHz after measuring the first level;
measuring a second level of PKM2 uptake in the cancer cells; and
determining if the patient is susceptible to treatment using alternating electric fields based on whether the first level is higher than the second level by at least 5%.

2. The method of claim 1, wherein the PKM2 probe comprises [18F]DASA-23 having the following structure:

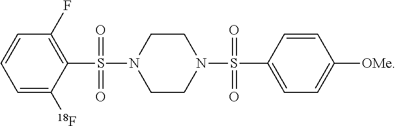 [SMILES: COc1ccc(S(=O)(=O)N2CCN(S(=O)(=O)c3c(F)cccc3[18F])CC2)cc1]

3. The method of claim 1, wherein the alternating electric fields have a frequency between 180 and 220 kHz.

4. The method of claim 1, wherein the cancer is glioblastoma.

5. A method of reducing a viability of cancer cells, comprising:
administering a PKM2 probe to a patient having cancer;
measuring a first level of PKM2 expression or uptake of the PKM2 probe in cancer cells from the patient;
exposing the cancer cells to alternating electric fields at a frequency between 100 and 500 kHz for a first interval of time after measuring the first level;
measuring a second level of PKM2 expression or uptake of the PKM2 probe in the cancer cells after the first interval of time; and
continuing exposing the cancer cells to alternating electric fields if the first level is higher than the second level by at least 5%.

6. The method of claim 5, wherein the PKM2 probe comprises [18F]DASA-23 having the following structure:

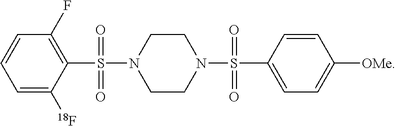 [SMILES: COc1ccc(S(=O)(=O)N2CCN(S(=O)(=O)c3c(F)cccc3[18F])CC2)cc1]

7. The method of claim 5, wherein the alternating electric fields have a frequency between 180 and 220 kHz.

8. The method of claim 5, wherein the cancer cells are glioblastoma cells.

9. A method of determining susceptibility of patient to treatment of cancer using alternating electric fields, comprising:
administering mannose labelled with an imaging probe to cells of patient having cancer;
measuring a first level of uptake of the mannose labelled with an imaging probe in cancer cells from the patient;
treating the cancer cells with alternating electric fields at a frequency between 100 and 500 kHz for a first interval of time after measuring the first level;
measuring a second level of uptake of the mannose labelled with an imaging probe in the cancer cells after the first interval of time; and
continuing treatment of the cancer cells using alternating electric fields if the first level is lower than the second level by at least 10%.

10. The method of claim 9, wherein the alternating electric fields have a frequency between 180 and 220 kHz.

11. The method of claim 9, wherein the cancer is glioblastoma.

12. A method of reducing a viability of cancer cells, comprising administering mannose to cancer cells of a patient having cancer and then exposing the cancer cells to alternating electric fields at a frequency between 100 and 500 kHz.

13. The method of claim 12, wherein the alternating electric fields have a frequency between 180 and 220 kHz.

14. The method of claim 12, wherein the cancer cells are glioblastoma cells.

15. A method of reducing a viability of cancer cells, comprising:
administering a PKM2 probe to a patient having cancer;
measuring a first level of PKM2 expression or uptake of the PKM2 probe in cancer cells from the patient;
exposing the cancer cells to alternating electric fields at a frequency between 100 and 500 kHz for a first interval of time after measuring the first level;
measuring a second level of PKM2 expression or uptake of the PKM2 probe in the cancer cells after the first interval of time; and
administering a chemotherapeutic agent to the cancer cells if the first level is higher than the second level by at least 5%.

16. The method of claim 15, wherein the chemotherapeutic agent is selected from the group consisting of tamoxifen, cisplatin, 5-fluorouracil (5-FU), and docetaxel.

17. The method of claim 16, wherein the chemotherapeutic agent is cisplatin.

18. The method of claim 15, further comprising continuing exposing the cancer cells to alternating electric fields.

19. The method of claim 15, wherein the cancer cells are glioblastoma cells.
